(19) United States
(12) Patent Application Publication
(10) Pub. No.: US 2009/0018653 A1
(43) Pub. Date: Jan. 15, 2009

(54) HYBRID AND PORTABLE POWER SUPPLIES FOR ELECTROLYTICALLY DETACHING IMPLANTABLE MEDICAL DEVICES

(75) Inventors: Mehran Bashiri, San Carlos, CA (US); Scott Merchel, Livermore, CA (US); Russell Ford, Palo Alto, CA (US)

Correspondence Address: VISTA IP LAW GROUP LLP, 12930 Saratoga Avenue, Suite D-2, Saratoga, CA, 95070, US

(73) Assignee: Boston Scientific Scimed, Inc., Maple Grove, MN (US)

(21) Appl. No.: 12/166,190

(22) Filed: Jul. 1, 2008

Related U.S. Application Data
(60) Provisional application No. 60/949,830, filed on Jul. 13, 2007.

(51) Int. Cl. A61F 2/02; H02B 1/24; A61B 17/58

(52) U.S. Cl. 623/11.11; 307/112; 606/99

(57) ABSTRACT

A medical system comprises a power supply coupled to an implant assembly, the power supply configured for detecting an energy delivery type of the implantable assembly and delivering electrical energy to the implant assembly in a mode corresponding to the detected energy delivery type, thereby electrolytically severing the joint. In another embodiment, a power supply is provided for use with a medical device having an elongated member and a terminal disposed on a proximal end of the elongated member. The power supply including power delivery circuitry, an electrical contact coupled to the power delivery circuitry, a port configured for receiving the proximal end of the elongated member, and an electrically insulative compliant member configured for urging the electrical terminal into contact with the electrical contact when the proximal end of the elongated member is received into the port.

### FIELD OF THE INVENTION

The invention relates generally to implantable devices (e.g., embolic coils, stents, and filters) having flexible electrolytic detachment mechanisms.

### BACKGROUND

Implants may be placed in the human body for a wide variety of reasons. For example, stents are placed in a number of different anatomical lumens within the body. They may be placed in blood vessels to cover vascular lesions or to provide patency to the vessels. Stents are also placed in biliary ducts to prevent them from kinking or collapsing. Grafts may be used with stents to promote growth of endothelial tissue within those vessels. As another example, vena cava filters can be implanted in the vena cava to catch thrombus sloughed off from other sites within the body and carried to the implantation site via the blood stream.

As still another example, vaso-occlusive devices are used for a wide variety of reasons, including for the treatment of intravascular aneurysms. An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality. Vaso-occlusive devices can be placed within the vasculature of the human body, typically via a catheter, either to block the flow of blood through a vessel making up that portion of the vasculature through the formation of an embolus or to form such an embolus within an aneurysm stemming from the vessel. The embolus seals and fills the aneurysm, thereby preventing the weakened wall of the aneurysm from being exposed to the pulsing blood pressure of the open vascular lumen.

One widely used vaso-occlusive device is a helical wire coil having windings, which may be dimensioned to engage the walls of the vessels. These coils typically take the form of soft and flexible coils having diameters in the range of 10-30 mils. Multiple coils will typically be deployed within a single aneurysm. There are a variety of ways of discharging vaso-occlusive coils into the human vasculature. In addition to a variety of manners of mechanically deploying vaso-occlusive coils into the vasculature of a patient, U.S. Pat. No. 5,122,136, issued to Guglielmi et al., describes an electrolytically detachable vaso-occlusive coil that can be introduced through a microcatheter and deployed at a selected location in the vasculature of a patient.

This vaso-occlusive coil is attached (e.g., via welding) to the distal end of an electrically conductive pusher wire. With the exception of a sacrificial joint just proximal to the attached embolic device, the outer surface of the pusher wire is coated with an ionically non-conductive material. Thus, the sacrificial joint will be exposed to bodily fluids when deployed within the patient. A power supply is used to apply a positive voltage to the pusher wire via the power supply relative to the ground return causes an electrochemical reaction between the sacrificial joint and the surrounding bodily fluid (e.g., blood). As a result, the sacrificial joint will dissolve, thereby detaching the vaso-occlusive coil from the pusher wire at the selected site.

Traditionally, monopolar pusher wires have been used to deliver vaso-occlusive devices into the patient, requiring the delivery of the electrical current from the power supply to the sacrificial joint via an electrical path along the pusher wire, and returning the electrical current from the sacrificial joint back to the power supply via an electrical path through the patient's body to a conductive patch or intravenous needle located on or in the patient. More recently, bipolar pusher wires have been developed, requiring the delivery of the electrical current from the power supply to the sacrificial joint via an electrical path along the pusher wire, and returning the electrical current from the sacrificial joint back to the power supply via another electrical path along the pusher wire.

While both monopolar and bipolar pusher wires can be used with success, different power supplies must currently be used; that is, due primarily to the different electrical return paths, a power supply designed to deliver current to a monopolar pusher wire cannot be used to deliver current to a bipolar pusher wire, and a power supply designed to deliver current to a bipolar pusher wire cannot be used to deliver current to a monopolar pusher wire. In addition, there are currently different types of monopolar pusher wires that are designed to operate only with specific power supplies. Thus, to maintain the flexibility of using different pusher wires, different power supplies must be stored in an operating room.

There, thus, remains a need to provide an improved power supply capable of being used with different types of pusher wires to electrolytically deliver implants within a patient.

### SUMMARY OF THE INVENTION

In accordance with an aspect of the inventions, a medical system is provided. The medical system comprises an implant assembly including an elongated pusher member, an implantable device mounted to the distal end of the pusher member, and an electrolytically severable joint disposed on the pusher member, wherein the implantable device detaches from the pusher member when the joint is severed. In one embodiment, the implantable device is a vaso-occlusive device.

The medical system further comprises a power supply coupled to the implant assembly. The power supply is configured for detecting an energy delivery type (e.g., a monopolar type or bipolar type) of the pusher member and delivering electrical energy to the implant assembly in a mode corresponding to the detected energy delivery type, thereby electrolytically severing the joint.

In one embodiment, the power supply is configured for detecting the energy delivery type of the implant assembly by delivering an electrical signal (e.g., an alternating current (AC) signal) to the implant assembly and measuring an electrical parameter (e.g., one indicative of an impedance) in response to the delivered electrical signal.

The power supply may be configured for conveying the electrical signal between two points on the proximal end of the pusher member. In this case, the power supply may be configured for detecting that the energy delivery type is a monopolar type if the measured electrical parameter indicates a short circuit between the two points, and for detecting that the energy delivery type is a bipolar type if the measured electrical parameter indicates a finite resistance between the two points.

The power supply may be further configured for informing a user of a faulty electrical connection if the measured electrical parameter indicates an open circuit between the two points. If a monopolar type is detected, the faulty electrical connection must be checked in another manner. For example, the power supply may further be configured for delivering another electrical signal between the proximal end of the pusher member and an external ground electrode, measuring another electrical parameter in response to the other delivered electrical signal, and informing a user of a faulty electrical condition if the other measured electrical parameter indicates an open circuit between the proximal end of the pusher member and the external ground electrode.

In another embodiment, the power supply is configured for detecting the energy delivery type of the implant assembly by detecting whether a ground electrode is mated with the power supply. For example, the power supply may be configured for detecting that the energy delivery type is a monopolar type if a mating of the ground electrode with the power supply is detected, and for detecting that the energy delivery type is a bipolar type if a mating of the ground electrode with the power supply is not detected.

In accordance with a further aspect of the inventions, a method of performing a medical procedure on a patient is provided. The method comprises delivering an implant assembly within a patient, e.g., occlude a vascular body, such as an aneurysm. The implant assembly includes an elongated pusher member, an implantable device (e.g., a vaso-occlusive device) mounted to a distal end of the pusher member, and an electrolytically severable joint disposed on the pusher member. The method further comprises coupling the implant assembly to a power supply and automatically detecting an energy delivery type (e.g., a monopolar type or a bipolar type) of the implant assembly. The energy delivery type may be detected in the same manner described above. The method further comprises delivering electrical energy from the power supply to the implant assembly in a mode corresponding to the detected energy delivery type, thereby electrolytically severing the joint and detaching the implantable device from the pusher member.

In accordance with a still further aspect of the invention, a power supply is provided. The power supply comprises a first negative electrical contact configured for being coupled to an external ground electrode, and a positive electrical contact and a first negative electrical contact configured for being coupled to an implant assembly having a pusher member and an electrolytically detachable implantable device. In an optional embodiment, the power supply further comprises a port configured for receiving the proximal end of the pusher member to place the positive electrical contact and the second negative electrical contact into contact with the proximal end of the pusher member.

The power supply further comprises power delivery circuitry configured for being selectively operated in a bipolar delivery mode and a monopolar delivery mode, wherein electrical energy (e.g., DC electrical energy) is conveyed between the positive electrical contact and the first negative electrical contact during the monopolar delivery mode, and electrical energy is conveyed between the positive electrical contact and the second negative electrical contact during the bipolar delivery mode. In one embodiment, the electrical energy is conveyed from the power delivery circuitry within the range of 0.1-10 milliampheres. In another embodiment, the electrical energy is conveyed from the power delivery circuitry within the range of 0.1-10 volts. The power delivery circuitry may include a constant current source, although in alternative embodiments, the power delivery circuitry includes a constant voltage source. The power supply may further comprise a power source electrically coupled to the power delivery circuitry.

In one embodiment, the power supply further comprises control circuitry configured for determining an energy delivery type of the implant assembly, and for directing the power delivery circuitry to convey the electrical energy between the positive electrical contact and the first negative electrical contact if the determined energy delivery type is a monopolar type, and for directing the power delivery circuitry to convey the electrical energy between the positive electrical contact and the second negative electrical contact if the determined energy delivery type is a bipolar type.

The power supply may further comprise detection circuitry, in which case, the control circuitry may be configured for determining the energy delivery type of the implant assembly by directing the detection circuitry to convey an electrical signal (e.g., an AC signal) between the positive electrical contact and the second negative electrical contact, and measuring an electrical parameter (e.g., one indicative of impedance) in response to the conveyed electrical signal. Determination of the energy delivery type can be performed based on the measured electrical parameter in the same manner as described above with respect to the medical system.

Alternatively, the control circuitry is configured for determining the energy delivery type of the implant assembly by directing the detection circuitry to detect a coupling between the ground electrode and the first negative electrical contact, wherein the control circuitry is configured for determining that the energy delivery type is a monopolar type if coupling of the ground electrode to the first negative electrical contact is detected, and for determining that the energy delivery type is a bipolar delivery mode if coupling of the ground electrode to the first negative electrical contact is not detected.

The power supply may optionally comprise a status indicator, in which case, the control circuitry may be configured for directing the status indicator to indicate a faulty electrical connection if the measured electrical parameter indicates an open circuit between the positive electrical contact and the second negative electrical contact. If a monopolar type is determined, the control circuitry may be further configured for directing the detection circuitry to convey another electrical signal between the positive electrical contact and the first negative electrical contact, and measuring another electrical parameter in response to the other conveyed electrical signal. In this case, the control circuitry may be configured for directing the status indicator to indicate a faulty electrical connection if the other measured electrical parameter indicates an open circuit between the positive electrical contact and the first negative electrical contact.

In performing the foregoing functions, the power supply may comprise a switch. For example, to switch between electrical energy delivery modes, the power supply may further comprise a switch having an input terminal coupled to a negative terminal of the power delivery circuitry and first and second output terminals respectively coupled to the first and second negative electrical contact. In this case, the control circuitry is configured for selectively operating the switch to couple the input terminal to the first output switch terminal during the conveyance of the other electrical signal, and to couple the input terminal to the second output terminal during the conveyance of the electrical signal. In switching between detection of electrical parameters between the two points on the proximal end of the pusher member and between the proximal end of the pusher member and the ground electrode, the switch (or another switch) has an input terminal coupled to a negative terminal of the detection circuitry, and first and second output terminals respectively coupled to the first and second negative electrical contacts. In this case, the control circuitry is configured for selectively operating the switch to couple the input terminal to the first output terminal prior to the conveyance of the other electrical signal, and to couple the input terminal to the second output terminal prior to the conveyance of the electrical signal.

In accordance with yet another aspect of the inventions, a power supply for use with a medical device having an elongated member and a terminal disposed on a proximal end of the elongated member is provided. The power supply comprises power delivery circuitry, and an electrical contact electrically coupled to the power delivery circuitry. In one embodiment, the power delivery circuitry is configured for delivering electrical energy to the electrical contact within the range of 0.1-10 milliampheres. In another embodiment, the power delivery circuitry is configured for delivering electrical energy to the electrical contact within the range of 0.1-10 volts. In still another embodiment, the power delivery circuitry may be configured for delivering direct current (DC) electrical energy to the electrical contact. In an optional embodiment, the power supply further comprises a printed circuit board on which the power delivery circuitry and electrical contact are mounted. In other embodiments, the power supply may further comprise a power source electrically coupled to the power delivery circuitry, an actuator configured for being manipulated by a user to convey electrical energy from the power delivery circuitry to the electrical contact, and another electrical contact configured for being coupled to a ground electrode.

The power supply further comprises a port configured for receiving the proximal end of the elongated member, and an electrically insulative compliant member (e.g., a compliant pad) configured for urging the electrical terminal into contact with the electrical contact when the proximal end of the elongated member is received into the port. In an optional embodiment, the power supply further comprises another electrical contact electrically coupled to the power delivery circuitry, in which case, the compliant member is further configured for urging another electrical terminal disposed on the proximal end of the elongated member into contact with the other electrical contact when the proximal end of the elongated member is received into the port.

In one embodiment, the port includes a funnel having a large diameter distal portion and a small diameter proximal portion, in which case, the electrical contact may be located proximal to the small diameter proximal portion. The port may further include a cylindrical tube in communication with the small diameter proximal portion of the funnel, in which case, the electrical contact may be located proximal to the cylindrical tube. In an optional embodiment, the power supply further comprises a gel material disposed within the port that seals the electrical contact from an external environment. The power supply may comprise a hand-held portable housing in which the port, the power delivery circuitry, the electrical contact, and the compliant member are conveniently carried.

In accordance with still another aspect of the inventions, a medical system is provided. The medical system comprises a medical device including an elongated member, an electrical terminal disposed on a proximal end of the elongated member, and at least one operative element (e.g., an electrolytically severable joint) disposed on a distal end of the elongated member in electrical communication with the electrical terminal. In an optional embodiment, the medical device further includes an implantable device (e.g., a vaso-occlusive device) configured for detaching from the distal end of the elongated member when the joint is severed.

The medical system further comprises a power supply including a port in which the proximal end of the elongated member is disposed, power delivery circuitry, an electrical contact, and an electrically insulative compliant member that urges the electrical terminal into contact with the electrical contact. In one embodiment, the medical device further includes another electrical terminal disposed on the proximal end of the elongated member, and the at least one operative element is in electrical communication with the other terminal. In this case, the power supply may include another electrical contact electrically coupled to the power delivery circuitry, and the compliant member may urge the other electrical terminal into contact with the other electrical contact. The medical system may further comprises an external ground electrode, in which case, the power supply further includes another electrical contact configured for being coupled to the ground electrode. The details of the power supply can be similar to those described above.

Other and further aspects and features of the invention will be evident from reading the following detailed description of the illustrated embodiments, which are intended to illustrate, not limit, the invention.

### BRIEF DESCRIPTION OF DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view of a medical system arranged in accordance with one embodiment of the invention, wherein the medical system particularly delivers a vaso-occlusive device into a patient using a monopolar electrolytic delivery means;

FIG. 2 is a plan view of a medical system arranged in accordance with another embodiment of the inventions, wherein the medical system particularly delivers a vaso-occlusive device into a patient using a bipolar electrolytic delivery means;

FIG. 3 is a perspective view of a power supply used in the medical systems of FIGS. 1 and 2;

FIG. 4 is a cross-sectional view of the power supply of FIG. 3 mated with a monopolar implant assembly used in the medical system of FIG. 1;

FIG. 5 is a cross-sectional view of the power supply of FIG. 3 mated with a bipolar implant assembly used in the medical system of FIG. 2;

FIG. 6 is a cross-sectional view of the power supply of FIG. 3, taken along the line 6-6;

FIGS. 7 and 8 are cross-sectional views illustrating a method of delivering a vaso-occlusive device within an aneurysm of the patient utilizing the medical systems of FIG. 1 or FIG. 2; and

FIG. 9 is a flow diagram illustrating one method used by the power supply of FIG. 3 to detect an energy delivery type of an implantable assembly and delivering electrical energy to the implantable assembly in accordance with a mode corresponding to the detected energy delivery type.

### DETAILED DESCRIPTION OF THE ILLUSTRATED EMBODIMENTS

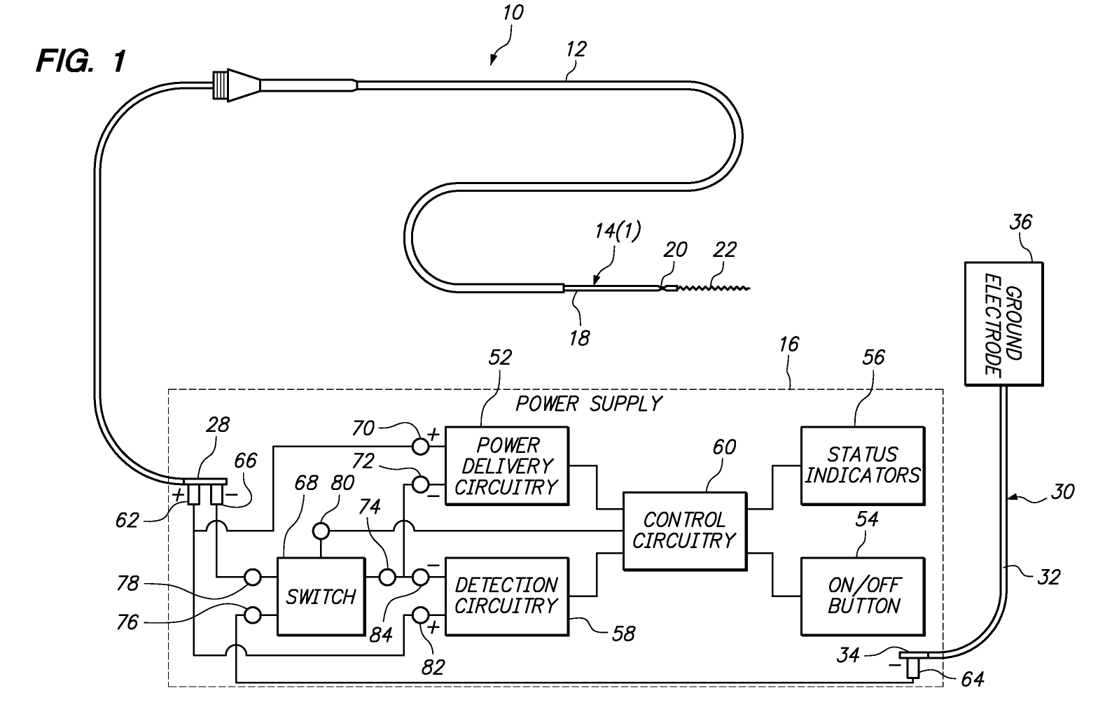

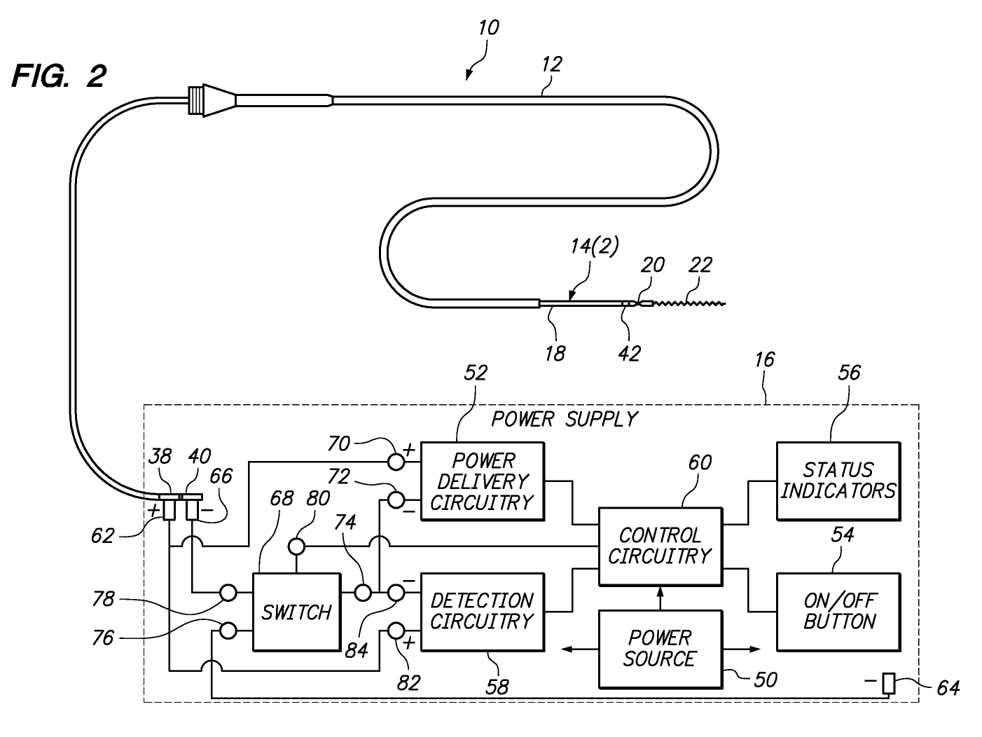

Referring generally to FIGS. 1 and 2, a medical system 10 constructed in accordance with one embodiment of the inventions will be described. The medical system 10 is used in vascular and neurovascular indications, and particularly in the treatment of aneurysms, such as cerebral aneurysms. The medical system 10 utilizes an electrolytic detachment means to deploy vaso-occlusive devices, such as helical coils, within an aneurysm. Alternatively, the medical system 10 can be utilized to deploy implantable devices other than vaso-occlusive devices. For example, the medical system 10 can alternatively be used to deploy stents and vena cava filters, which are described in further detail in U.S. Pat. No. 6,468,266, which is expressly incorporated herein by reference.

To this end, the medical system 10 generally comprises a delivery catheter 12 that can be intravenously introduced within a patient to access a target site within the vasculature, an implant assembly 14 that can be slidably disposed within the delivery catheter 12, and an electrical power supply 16 that can supply electrical energy to the implant assembly 14 to effect the electrolytic detachment process.

The delivery catheter 12 includes an elongate, flexible, tubular member 13 composed of a suitable polymeric material and optionally reinforced with a coil or braid to provide strength or obviate kinking propensities. The delivery catheter 12 further includes a lumen (not shown) through which the implant assembly 14 can be selectively located. The delivery catheter 12 further includes a pair of radiopaque markers 15 disposed on the distal end of the tubular member 13 to allow visualization of the delivery catheter 12 relative to the vaso-occlusive implant 22. The delivery catheter 12 further includes a proximal fitting 17 disposed on the proximal end of the tubular member 13 for introduction of the implant assembly 14, as well as for the optional introduction of dyes or treatment materials.

The implant assembly 14 includes a pusher member 18, an electrolytically severable joint 20, and a vaso-occlusive device 22 that detaches from the distal end of the pusher member 18 when the joint 20 is electrolytically severed. The pusher member 18 typically includes an electrically conductive core (not shown) that provides the pusher member 18 with the necessary tensile and columnar strength, an electrically insulative covering (not shown), and flexible coils (shown) that increase the flexibility of the pusher member 18 at its distal end.

Significantly, the power supply 16 is configured for being used with different types of implantable assemblies 14, some of which may take the form of a monopolar implant assembly 14(1) (shown in FIG. 1), which uses monopolar electrolytic means to detach the vaso-occlusive device 22 from the pusher member 18 at the severable joint 20, and some of which take the form of a bipolar implant assembly 14(2) (shown in FIG. 2), which uses bipolar electrolytic means to detach the vaso-occlusive device 22 from the pusher member 18 at the severable joint 20.

The monopolar implant assembly 14(1) includes a single terminal 28 (shown mated with the power supply 16) disposed on the proximal end of the pusher member 18. The single terminal 28 may simply be formed on the proximal end of the pusher member 18 by exposing the underlying core wire. The single terminal 28 serves as a positive terminal that is electrically coupled to the severable joint 20. In this case, the system 10 includes a separate return electrode assembly 30, which includes a cable 32, a negative terminal 34 disposed on the proximal end of the cable 32, and an external return electrode 36 in the form of a ground patch electrode or a ground needle electrode disposed on the distal end of the cable 32, which can be placed into contact with the patient's tissue remote from the implant assembly 14(1). Thus, a monopolar patient circuit can be formed between the severable joint 20 at the distal end of the pusher member 18 and the return electrode 32 remotely located from the severable joint 20. An optional intermediate return electrode (not shown) can be carried by the distal end of the pusher member 18 to enhance the monopolar patient circuit.

In contrast, the bipolar implant assembly 14(2) includes positive and negative terminals 38, 40 disposed on the proximal end of the pusher member 18 (shown mated with the power supply 16), and a return (ground) electrode 42 carried by the distal end of the pusher member 18 adjacent to the severable joint 20. The positive terminal 32 is electrically coupled to the severable joint 20, whereas the negative terminal 34 is electrically coupled to the return electrode. Thus, a bipolar patient circuit can be formed between the severable joint 20 and the return electrode at the distal end of the pusher member 18.

In either of the monopolar or bipolar arrangements, the severable joint 20 serves as an anode, and the return electrode or ground electrode serves as a cathode. In both of the monopolar and bipolar arrangements, the positive terminals 28, 38 are typically electrically coupled to the severable joint 20 via a stainless steel, or otherwise electrically conductive, core wire (not shown) that extends within and provides the necessary tensile and columnar strength for the pusher member 18. In the monopolar arrangement, however, the entire proximal end of the pusher member 18 will typically be uninsulated to expose the underlying core wire, which will serve at the positive terminal 28. Further details discussing various exemplary constructions of monopolar and bipolar implant assemblies are disclosed in U.S. Patent No. 60/939,032 (Attorney Docket No. 06-01517 (US01), which is expressly incorporated herein by reference.

The power supply 16 is configured for being selectively operated either in a monopolar energy delivery mode by conveying electrical energy between the severable joint 20 of the monopolar implant assembly 14(1) and the external ground electrode 36, thereby electrolytically severing the joint 20 in order to detach the vaso-occlusive device 22 from the pusher member 18, and in a bipolar energy delivery mode by conveying electrical energy between the severable joint 20 and return electrode 42 of the bipolar implant assembly 14(2), thereby electrolytically severing the joint 20 in order to detach the vaso-occlusive device 22 from the pusher member 18. The electrical energy conveyed to either of the implant assemblies 14 can have any waveform that induces electrolysis between the joint 20 and the surrounding body fluids, but preferably takes the form of direct current (DC) electrical energy.

In order to place the power supply 16 in the proper electrical current delivery mode (either bipolar or monopolar) consistent with the type of implant assembly intended to be used, the power supply 16 is configured for detecting the electrical energy delivery type of the implant assembly 14 that is currently mated with the power supply 16, and then delivering electrical energy to the implant assembly 14 in the mode corresponding to the detected energy delivery type.

The power supply 16 detects the energy delivery type of the implant assembly 14 by delivering an electrical signal to the implant assembly 14 and measuring an electrical parameter in response to the delivered signal. In the illustrated embodiment, the delivered signal is an alternating current (AC) signal, and the measured electrical parameter is an impedance, although the delivered signal may be, e.g., a constant direct current (DC) signal, and the measured electrical parameter may be, e.g., an voltage magnitude or current magnitude.

In the illustrated embodiment, the power supply 16 is configured for delivering the electrical signal between two points at the proximal end of the pusher member 18, and measuring the impedance between the two points to detect the energy delivery type of the implant assembly 14. Notably, if the monopolar implant assembly 14(1) is mated with the power supply 16, the two points between which the electrical signal is conveyed will be across a length of the positive terminal 28 of the implant assembly 14(1) (i.e., between points along the exposed core wire), thereby creating a short circuit across a relatively short electrical path. As a result, the measured impedance measured across the two points in this case will be approximately zero, indicating a short circuit between the two points.

In contrast, if the bipolar implant assembly 14(2) is mated with the power supply 16, the two points between which the electrical signal is conveyed will be across the positive and negative terminals 38, 40 of the implant assembly 14(2). As a result, the impedance measured across the two points will be finite, since the electrical path between the positive and negative terminals 38, 40 will extend along the entire length of the pusher member 18, through the blood between the severable joint 20 and return electrode 42 located at the distal end of the pusher member 18, and back along the entire length of the pusher member 18, thereby indicating the existence of a resistive load between the two points.

Thus, based on the impedance measurements, the power supply 16 will be capable of identifying the type of implant assembly, and in particular, whether the implant assembly 14 currently mated with the power supply 16 requires bipolar delivery or monopolar delivery of the electrical current. That is, the power supply 16 will detect the currently mated implant assembly 14 as being monopolar if the measured impedance is approximately zero, and as being bipolar if the measured impedance is a finite value below a threshold.

Notably, the patient circuit, whether monopolar or bipolar, will not properly function if the current delivery mode of the implant assembly 14 is mis-detected (i.e., a bipolar implant assembly is detected as a monopolar implant assembly, or a monopolar implant assembly is detected as a bipolar implant assembly), the power supply 16 is not compatible with the mated implant assembly 14, the implant assembly 14 or ground electrode assembly 30 is not properly mated with the power supply 16, or there is a broken electrical connection within the implant assembly 14 or ground electrode assembly 30.

Thus, in the illustrated embodiment, the power supply 16 is also configured for checking proper functioning of the patient circuit by conveying an electrical signal between the positive terminal 28 and the negative terminal 34 if a monopolar implant assembly 14(1) is initially detected, or between the positive and negative terminals 38, 40 if a bipolar implant assembly 14 is initially detected, and then measuring the impedance. In the case of a properly functioning patient circuit, the measured impedance will be finite (i.e., indicating a resistive load between the positive and negative terminals 28, 34 or between the positive and negative terminals 38, 40), and in the case of an improperly functioning circuit, the measured impedance will be infinite (i.e., indicating an open circuit between the positive and negative terminals 28, 34 or between the positive and negative terminals 38, 40).

Thus, if the measured impedance indicates an open circuit between the two points on the proximal end of the pusher member 18, the power supply 16 is configured for indicating a faulty electrical connection to the user. In the case where a monopolar implant assembly is detected, the power supply 16 is configured for delivering another electrical signal (e.g., an AC signal) between the proximal end of the pusher member 18 and the ground electrode 36, measuring another electrical parameter (in particular, impedance) in response to the delivery of the other electrical signal, and indicating a faulty electrical connection if the other measured impedance indicates an open circuit between the proximal end of the pusher member 18 and the ground electrode 36. The power supply 16 may optionally be configured for entering a check mode that informs the user to check the connections if the impedance measured between the two points on the proximal end of the pusher member 18 or the impedance measured between the proximal end of the pusher member and the ground electrode 36 indicates an open circuit.

The power supply 16 may assume that a measured impedance indicates an open circuit if it is greater than a threshold value. Notably, the impedance of a properly functioning monopolar patient circuit, which traverses the tissue extending from the severable joint 20 at the distal end of the pusher member 18 to the remote ground electrode 36, will be greater than the impedance of a properly functioning bipolar patient circuit, which only traverses the tissue located between the severable joint 20 and return electrode 42 at the distal end of the pusher member 18. Thus, the power supply 16 may prevent the delivery of electrical energy to a mated implant assembly 14 based on different threshold values. For example, if the measured impedance between the two points on the proximal end of the pusher member 18 is above a threshold value (e.g., greater than 500 ohms), or if the measure impedance between the proximal end of the pusher member 18 and the ground electrode 36 is above a threshold value (e.g., greater than 200 ohms), the power supply 16 may be configured to prevent the delivery of electrical energy to the implant assembly 14.

In an alternative embodiment, the power supply 16 is configured for detecting the energy delivery type of the implant assembly 14 by detecting wither the ground electrode 36 is mated with the power supply 16. That is, mating or not mating of the ground electrode 36 to the power supply 16 is indicative of the type of implant assembly 14 mated to the power supply 16. Thus, in this case, the power supply 16 is configured for detecting that the energy delivery type of the mated implant assembly 14 is a monopolar type if the ground electrode 36 is mated with the power supply 16, and for detecting that the energy delivery type of the mated implant assembly 14 is a bipolar type if the ground electrode 36 is not mated with the power supply 16. Of course, this methodology of detecting the energy delivery type of mated implant assembly 14 relies heavily on the intentions of the user, and thus, may not be as suitable as the previously described detection methodology if the user errs by mating the bipolar implant assembly 14(2) and ground electrode 36 to the power supply 16 or mating the monopolar implant assembly 14(1) without mating the ground electrode 36 to the power supply 16.

Having described the function of the power supply 16, its components will now be described. The power supply 16 comprises a power source 50 configured for supplying power at the necessary voltage levels to the components of the power supply 16 and power delivery circuitry 52 configured for delivering the electrical energy necessary to electrolytically detach the vaso-occlusive device 22 of the implant assembly 14 coupled to the power supply 16.

The power source 50 may comprise conventional components, such as one or more batteries (e.g., standard 9V alkaline batteries or a AAA battery), and one or more voltage regulators (not shown) for converting the voltage provided by the output of the battery or batteries to different voltages that can be utilized by the components of the power supply 16.

The power delivery circuitry 52 may comprise an output drive circuit (not shown), which may take the form of a constant current source that will apply as much voltage as necessary to maintain the required current, a current-enable circuit (not shown) for turning the output drive circuit on, a current adjustment circuit (not shown) for adjusting the magnitude of the current output by the output drive circuit, and a patient isolation relay (not shown) that can be energized to decouple the implant assembly 14 from the output drive circuit during the power up diagnostics, after the vaso-occlusive device 22 is detached, or if a failure occurs during a procedure. In the illustrated embodiment, the current output by the power delivery circuitry 52 is a constant direct current (DC) waveform, although other waveforms that induce electrolysis can be used. The electrical energy conveyed from the power delivery circuitry 52 is preferably within the range of 0.1-10 milliampheres. If, alternatively, a voltage source is used, the electrical energy conveyed from the power delivery circuitry 52 is preferably within the range of 0.1-10 volts.

The power supply 16 further comprises a power on/off actuator 54 configured for alternately activating and deactivating the power supply 16, and status indicators 56 for providing the status of the power supply 16 and electrolytic detachment process. The on/off actuator 54 may take the form of a conventional push button toggle switch that a user can alternately depress to activate and deactivate the power delivery circuitry 52. That is, initial actuation of the on/off actuator 54 will cause the power delivery circuitry 52 to deliver electrical energy to the mated implant assembly 14, and subsequent actuation of the on/off actuator 54 will cause the power delivery circuitry 52 to cease delivering electrical energy to the mated implant assembly 14. The status indicators 56 may take the form of any visible and/or audible indicators that provides status, such as low battery, power delivery state, detachment of the vaso-occlusive device 22, and misconnection within the patient circuit.

The power supply 16 further comprises detection circuitry 58 configured for detecting an electrical parameter indicative of a detachment event between the vaso-occlusive device 22 and the pusher member 18. In performing these functions, the detection circuitry 58 may comprises an alternating current (AC) signal generator (not shown) that superimposes or otherwise generates an AC signal in conjunction with the DC current generated the power delivery circuitry 52, and an AC-to-DC rectifier and peak detector (not shown) that measures the magnitude of the AC signal and outputs a DC signal. The detection circuitry 58 may also comprise a DC monitor for measuring the magnitude of the DC signal output by the power delivery circuitry 52.

The power supply 16 further comprises control circuitry 60 configured for monitoring and controlling the vital functions of the power supply 16. The control circuitry 60 may comprise a microcontroller that performs such functions, as controlling the current enable circuit of the power delivery circuitry in response to user operation of the on/off actuator 54, controlling the current adjust circuit and patient isolation relay of the power delivery circuitry under various conditions, determining detachment of the vaso-occlusive device 22 based on the feedback from the detection circuitry 58, managing the status indicators, running self-diagnostics, etc. The control circuitry 60 may be implemented in firmware, hardware, software, or in combination thereof.

With respect to the conventional functions performed by the power supply 16, much of the functional details of the foregoing components are described in U.S. Pat. Nos. 5,669,905 and 6,397,850, which are expressly incorporated herein by reference. However, as discussed above, the power supply 16 has the capability of delivering electrical energy to the implant assembly 14 in either a monopolar mode or a bipolar mode to detach the vaso-occlusive device 22 from the pusher member 18.

To this end, the power supply 16 is equipped with electrical contacts that can accommodate both monopolar and bipolar implant assemblies 14. In particular, the power supply 16 has a positive electrical contact 62 configured for directly contacting the positive terminal 28 of the monopolar implant assembly 14(1) (FIG. 1) or the positive terminal 38 of the bipolar implant assembly 14(2) (FIG. 2), a first negative electrical contact 64 configured for directly contacting the negative terminal 34 of the ground electrode assembly 30 (FIG. 1), and a second negative electrical contact 66 configured for being electrically coupled to the negative terminal 40 of the bipolar implant assembly 14(2) (FIG. 2). For the purposes of this specification, the terms “positive” and “negative” with respect to a terminal or contact is relative and merely means that the positive terminal or contact has a greater voltage potential than that of the negative terminal or contact.

To ensure compatibility between the power supply 16 and the bipolar implant assembly 14(2), the positive electrical contact 62 and the second negative electrical contact 66 are preferably located and spaced, such that they respectively contact the positive and negative terminals 38, 40 of the implant assembly 14(2) when mated with the power supply 16. Because the entire proximal end of the pusher member 18 of the monopolar implant assembly 14(1) serves as the positive terminal 28, the positive electrical contact 62 of the power supply 16 will naturally be in contact with the positive terminal 28 without much concern with the positioning of the positive electrical contact 62 when the pusher member 18 of the implant assembly 14(2) is mated with the power supply 16.

The control circuitry 60 is configured for selectively directing the power delivery circuitry 52 to be operated in a monopolar delivery mode, wherein the electrical energy is conveyed between the positive electrical contact 62 and the first negative electrical contact 64, and a bipolar delivery mode, wherein the electrical energy is conveyed between the positive electrical contact 62 and the second negative electrical contact 66.

To this end, the power supply 16 further comprises a switch 68 through which the power delivery circuitry 52 can be selectively coupled between the positive electrical contact 62 and the first negative electrical contact 64 or between the positive electrical contact 62 and the second negative electrical contact 66. In particular, the power delivery circuitry 52 comprises a positive terminal 70 coupled to the positive electrical contact 62, and a negative terminal 72 coupled to the first and second negative contacts 64, 66 through the switch 68.

That is, the switch 68 has an input terminal 74 coupled to the negative terminal 72 of the power delivery circuitry 52, a first output terminal 76 coupled to the first negative electrical contact 64, and a second output terminal 78 coupled to the second negative electrical contact 66. The switch 68 also has a control terminal 80 to which the control circuitry 60 is coupled. Thus, the control circuitry 60 can send control signals to the switch 68 to selectively couple the input terminal 74 of the switch 68 (and thus, the negative terminal 72 of the power delivery circuitry 52) to either the first output terminal 76 of the switch 68 (and thus, the first negative electrical contact 64), thereby placing the power delivery circuitry 52 in a monopolar mode, or to the second output terminal 78 of the switch 68 (and thus, the second negative electrical contact 66), thereby placing the power delivery circuitry 42 in a bipolar mode.

As such, under control of the control circuitry 60 (and in response to actuation of the on/off actuator 54), the power delivery circuitry 52 can deliver electrical energy between the positive electrical contact 62 and the first negative electrical contact 64, and thus, between the positive terminal 28 of the monopolar implant assembly 14 and the negative terminal 34 of the ground electrode assembly 30, to detach the vaso-occlusive device 22 from the pusher member 18, or the power delivery circuitry 52 can deliver electrical energy between the positive electrical contact 62 and the second negative electrical contact 66, and thus, between the positive and negative terminals 38, 40 of the bipolar implant assembly 14(2), to detach the vaso-occlusive device 22 from the pusher member 18.

In the illustrated embodiment, the control circuitry 60 is configured for determining an energy delivery type of the implant assembly 14, and the control circuitry 60 is configured for directing the power delivery circuitry 52 to delivery electrical energy between the positive electrical contact 62 (and thus the positive electrical terminal 28 of the monopolar implant assembly 14(1)) and the first negative electrical contact 64 (and thus the negative electrical terminal 34 of the ground electrode assembly 30) if the determined energy delivery type is a monopolar type, and for directing the power delivery circuitry 52 to delivery electrical energy between the positive electrical contact 62 and the second negative electrical contact 66 (and thus the positive and negative electrical terminals 38, 40 of the bipolar implant assembly 14(2)) if the detected energy delivery type is a bipolar type.

To determine the energy delivery mode of the implant assembly 14, the control circuitry 60 is configured for directing the detection circuitry 58 to convey an electrical signal (e.g., an AC signal) to the positive electrical contact 62, and to measure an electrical parameter in response to the conveyed electrical signal. In particular, the electrical signal is conveyed between the positive and negative electrical contacts 62, 66 (and thus between two points across the proximal end of the pusher member 18), and the measured electrical parameter is indicative of the impedance between the electrical contacts 62, 66. The control circuitry 60 is configured for determining that the energy delivery type of the implant assembly 14 is monopolar if the impedance between the positive electrical contact 62 and the second electrical contact 66 is approximately zero, indicating a short circuit between the electrical contacts 62, 66, and for determining that the energy delivery type of the implant assembly 14 is bipolar if the impedance between the positive electrical contact 62 and the second negative electrical contact 66 is finite, indicating a resistive load between the electrical contacts 62, 66.

The control circuitry 60 is further configured for determining whether the patient circuit is properly functioning, and for directing one or more of the status indicators 56 to indicate a faulty electrical connection if the patient circuit is not properly functioning. In particular, if the impedance between the positive electrical contact 62 and the second negative electrical contact 66 indicates an open circuit (i.e., the impedance value is greater than a threshold value, e.g., 500 ohms), the control circuitry 60 is configured for directing one or more of the status indicators 56 to inform the user of a faulty electrical connection within the bipolar patient circuit. If a monopolar energy delivery type is detected, the functioning of the monopolar patient circuit must be checked. In this case, the control circuitry 60 is configured for directing the detection circuitry 58 to convey another electrical signal (e.g., an AC signal) between the positive electrical contact 62 (and thus the positive electrical terminal 38 of the monopolar implant assembly 14(1)) and the first negative electrical contact 64 (and thus the negative electrical terminal 34 of the ground electrode assembly 30), and measuring another electrical parameter indicative of the impedance between the positive electrical contact 62 and the first negative electrical contact 64. If the impedance between the positive electrical contact 62 and the first negative electrical contact 64 indicates an open circuit (i.e., the impedance value is greater than a threshold value, e.g., 2000 ohms), the control circuitry 60 is configured for directing one or more of the status indicators 56 to inform the user of a faulty electrical connection within the monopolar patient circuit.

To enable the power supply 16 to detect the type of the implant assembly 14 by measuring the impedance across the proximal end of the pusher member 18 of the implant assembly 14 currently mated to the power supply 16, and to check the functioning of both the monopolar patient circuit and the bipolar patient circuit, the detection circuitry 58, like the power delivery circuitry 52, is selectively coupled between the positive electrical contact 62 and the first negative electrical contact 64 or between the positive electrical contact 62 and the second negative electrical contact 66 via the switch 68.

In particular, the detection circuitry 58 comprises a positive terminal 82 coupled to the positive electrical contact 62, and a negative terminal 84 coupled to the first and second negative contacts 64, 66 through the switch 68. That is, the input terminal 74 of the switch 68 is coupled to the negative terminal 84 of the detection circuitry 58. Thus, the control circuitry 60 can send control signals to the switch 68 to selectively couple the input terminal 74 of the switch 68 (and thus, the negative terminal 84 of the detection circuitry 58) to either the first output terminal 76 of the switch 68 (and thus, the first negative electrical contact 64) or the second output terminal 78 of the switch 68 (and thus, the second negative electrical contact 66).

In this manner, the detection circuitry 58 is configured for measuring an electrical parameter indicative of the impedance between the positive electrical contact 62 and the first negative electrical contact 64 (so that the functioning of the patient circuit in the monopolar mode can be checked) or between the positive electrical contact 62 and the second negative electrical contact 66 (so that the implant assembly type can be detected or the functioning of the patient circuit in the bipolar mode can be checked) in response to the conveyed electrical signal.

In the alternative embodiment where the power supply 16 determines the energy delivery type of the mated implant assembly 14 based on whether the ground electrode 36 is mated to the power supply 16, the control circuitry 60 is configured for directing the detection circuitry 58 to detect a coupling of the ground electrode 36 to the first negative electrical contact 64. The control circuitry 60 is configured for determining the energy delivery type of the implant assembly 14 by directing the detection circuitry 58 to detect a coupling between the ground electrode 36 and the first negative electrical contact 64. Coupling of the ground electrode 36 to the first negative electrical contact 64 can be detected using suitable means, such as measuring an impedance between two contacts (not shown) between which the terminal 34 of the ground assembly 30 contacts when mated with the power supply 14, with a short circuit indicating that coupling between the ground electrode 36 and the first negative electrical contact. If a coupling of the ground electrode 36 to the first negative electrical contact 64 is detected, the control circuitry 60 is configured for determining that the energy delivery type is a monopolar type, and if a coupling of the ground electrode 36 to the first negative electrical contact 64 is not detected, the control circuitry 60 is configured for determining that the energy delivery type is a bipolar delivery mode.

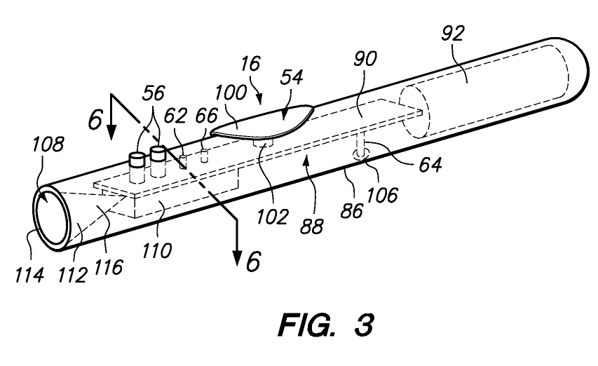

Referring now to FIGS. 3-6, the physical components of the power supply 16 will be described. The power supply 16 comprises a portable hand-held housing 86 having a cavity 88 that encloses the components of the power supply 16. In the illustrated embodiment, the housing 86 is composed of a suitable medical-grade rigid material, such as polycarbonate, and has a cylindrical shape that can be easily held within a hand—much like a pen.

The power supply 16 further comprises a printed circuit board 90 configured for carrying the electronic components, including the power on/off actuator 54, the positive electrical contact 62, the first and second negative electrical contacts 64, 66, and status indicators 56. The power source 50 (except for the battery), power delivery circuitry 52, detection circuitry 58, control circuitry 60, and switch 68, although not shown in FIGS. 3-6, are also carried by the printed circuit board 90. The power supply 16 further comprises a battery 92 (as part of the power source 50 shown in FIGS. 1 and 2), the positive terminal 94 of which is in direct electrical contact with a power terminal 96 mounted to a printed circuit board 90, and the negative terminal (not shown) of which is indirectly coupled to a negative terminal (not shown) of the printed circuit board 90 via a cable (not shown). The battery 92 may take the form of any suitable low profile battery, such as a AAA battery, and is capable of providing enough power to effect multiple detachments. The cost of the power supply 16 may be low enough to economically allow its disposal after a single clinical procedure.

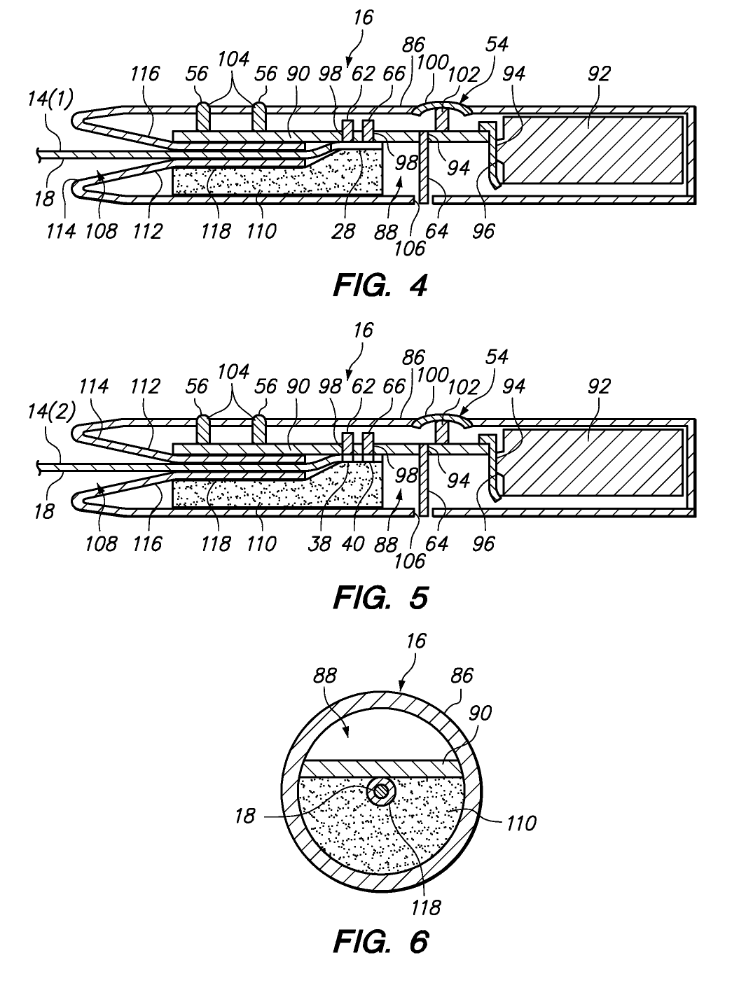

In the illustrated embodiment, the electrical contacts 62-66 are fixably mounted within through-holes 98 (shown in FIGS. 4 and 5) within the printed circuit board 90, and are electrically coupled to the power delivery circuitry 52 carried by printed circuit board 90. The power on/off actuator 54 includes a button 100 mounted on the exterior of the housing 86 and a switch 102 directly mounted on the printed circuit board 90. Depression of the button 100 by a user manipulates the switch 102 to deliver or cease delivery of electrical energy from the power delivery circuitry 52 between the positive electrical contact 62 and the first or second negative electrical contacts 64, 66 in the manner described above with respect to FIGS. 1 and 2. The status indicators 56 take the form of light emitting diodes (LEDs), which as discussed above, can indicate status to the user, such as low battery, power delivery state, detachment of the vaso-occlusive device 22, and misconnection within the patient circuit. The status indicators 56 are exposed through apertures 104 (shown in FIGS. 4 and 5) formed within the housing 86.

The power supply 16 comprises a ground port 106 configured for receiving the proximal end of the ground cable 32 (not shown in FIGS. 3-6), so that the negative terminal 34 of the ground electrode assembly 30 is placed into contact with the first negative electrical contact 64. The power supply 16 further comprises a port 108 configured for receiving the proximal end of the pusher member 18 and an electrically insulative compliant member 110 configured for urging the positive terminal 28 of the monopolar implant assembly 14(1) into firm electrical contact with the positive electrical contact 62 and second negative electrical contact 66 affixed to the printed circuit board 90, or for respectively urging the positive and negative terminals 38, 40 of the bipolar implant assembly 14(2) into firm electrical contact with the positive electrical contact 62 and second negative electrical contact 66 affixed to the printed circuit board 90.

The compliant member 110 may be composed of any electrically insulative, resilient material. In the illustrated embodiment, the compliant member 110 takes the form of a compliant pressure pad disposed directly beneath and in contact with the lower surface of the printed circuit board 90, so that the proximal end of the pusher member 18 is advanced between the upper surface of the compliant pad 110 and the lower surface of the printed circuit board 90. The positive electrical contact 62 and second negative electrical contact 66 are located just proximal to the port 108, so that as proximal end of the pusher member 18 exits the port 108 in the proximal direction, the terminal(s) located on the pusher member 18 come into contact with the positive electrical contact 62 and second negative electrical contact 66.

The port 108 is shaped in a manner that guides the proximal end of the pusher member 18 into alignment with the positive electrical contact 62 and second negative electrical contact 66. In particular, the port 108 includes a funnel 112 having a large diameter distal portion 114 and a small diameter proximal portion 116, and a cylindrical tube 118 in communication with the small diameter proximal portion 116 of the funnel 112. Thus, as the proximal end of the pusher member 18 is introduced into the port 108, it is funneled into the cylindrical tube 118, which then guides the proximal end of the pusher member 18 into aligned with the electrical contacts 62, 66. In the illustrated embodiment, the cylindrical tube 118 is embedded within the compliant pad 110, as best shown in FIG. 6, so that the cylindrical tube 118 does not hinder firm contact between the bottom surface of the printed circuit board 90 and the compliant pad 110.

It can be appreciated that the interaction between the port 108 and the compliant pad 110 facilitates coupling between the implant assembly 14 and the power supply 16 simply by inserting the pusher member 18 into the port 108. Notably, this arrangement allows different types and sizes of pusher members 18 to be mated with the power supply 16. In an optional embodiment, the power supply 16 further comprises a gel material (not shown) disposed within the port 108 to seal the electrical contacts 62, 66 from the external environment, thereby reducing the chance that liquid and/or solid contaminants will adversely affect detachment reliability.

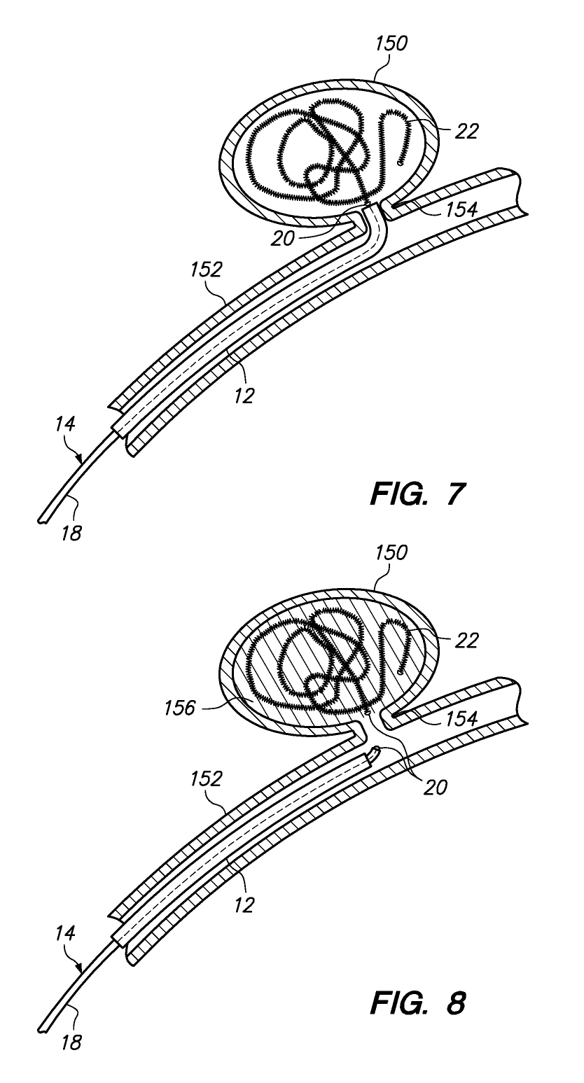

Having described the function and structure of the medical system 10, its operation in performing a medical procedure, and in particular implanting the vaso-occlusive device 22 within an aneurysm 150 of a patient, will now be described with reference to FIGS. 7 and 8. The process of implanting a vaso-occlusive device within a patient is typically practiced under fluoroscopic control with local anesthesia.

With reference to FIG. 7, the delivery catheter 12 is introduced within the patient via an entry point, such as the groin, and positioned within a blood vessel 152, with the tip of the catheter 12 residing within or adjacent a neck 154 of the aneurysm 150. The implant assembly 14 is then inserted within the delivery catheter 12 and advanced until the vaso-occlusive device 22 is disposed within the aneurysm 150. The implant assembly 14 is then coupled to the power supply 16. If the implant assembly 14 is monopolar, the ground electrode assembly 30 will be coupled to the power supply 16, as illustrated in FIG. 1, and if the implant assembly 14 is bipolar, the ground electrode assembly 30 will not be coupled to the power supply 16, as illustrated in FIG. 2. If the ground electrode 36 takes the form of a patch electrode, it can be applied to the skin of the patient, e.g., on the shoulder. If the ground electrode 36 takes the form of a needle electrode, it can be percutaneously inserted into the patient, e.g., in the groin.

With reference to FIG. 8, electrical energy is delivered from the power supply 16 to the implant assembly 14 via actuation of the on/off power actuator 54 to severe the junction 20 at the distal end of the pusher member 18, thereby detaching the vaso-occlusive device 22 from the pusher member 18. The occlusion of the vaso-occlusive device 22 forms a coagulum 156 within the aneurysm 150, thereby eliminating the danger that the aneurysm 150 will rupture.

In the case of a monopolar implant assembly 14(1), the electrical energy will be delivered between the severable joint 20 and the ground electrode 36 to electrolytically detach the vaso-occlusive device 22 from the pusher member 18. In the case of a bipolar implant assembly 14(2), the electrical energy will be delivered between the severable joint 20 and the return electrode 42 to electrolytically detach the vaso-occlusive device 22 from the pusher member 18. After the vaso-occlusive device 22 is detached, the pusher member 18 is removed from the delivery catheter 12. Additional vaso-occlusive devices may be implanted within the aneurysm 150 by introducing additional implant assemblies through the delivery catheter 12 and electrolytically detaching the vaso-occlusive devices within the aneurysm 150.

Significantly, before the on/off power actuator 54 is actuated, or immediately after the actuation of the on/off power actuator 54, the energy delivery type of the implant assembly 14 is automatically detected, and the electrical energy is delivered from the power supply to the implant assembly 14 in a mode corresponding to the detected energy delivery type, thereby electrolytically severing the joint 20 and detaching the implantable device 22 from the pusher member 18.

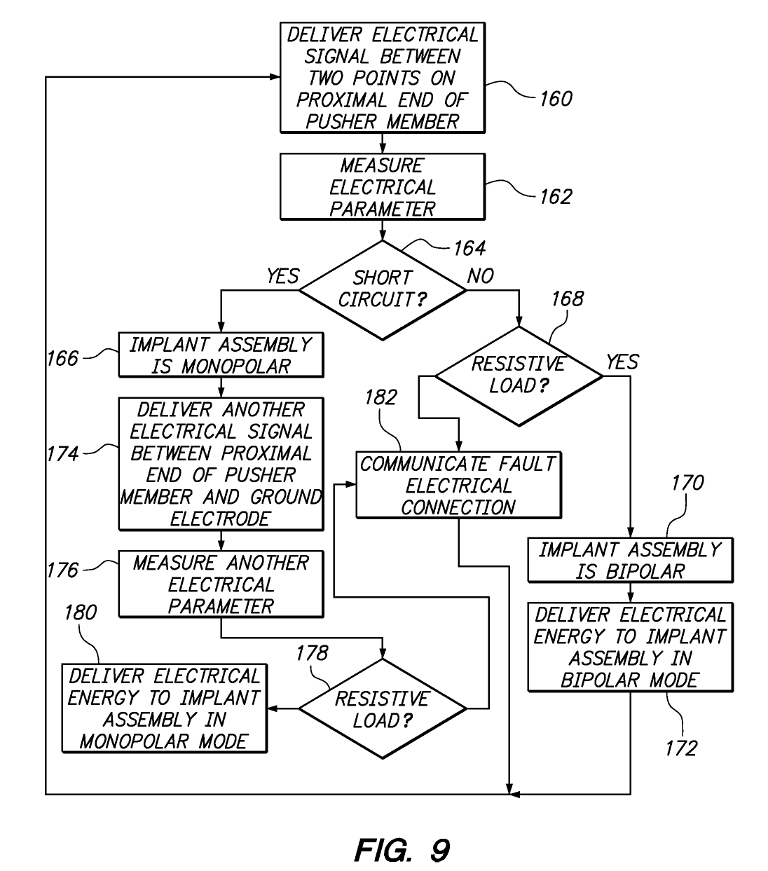

In particular, and with reference to FIG. 9, an electrical signal is delivered between two points on the proximal end of the pusher member 18 (step 160), and an electrical parameter is measured in response to the delivered electrical signal (step 162). If the measured electrical signal indicates a short circuit between the two points (step 164), the energy delivery type is detected as a monopolar type (step 166). If the measured electrical signal indicates a resistive load between the two points (step 168), the energy delivery type is detected as a bipolar type (step 170), and electrical energy is delivered to the implant assembly 14 in a bipolar mode to electrolytically detach the vaso-occlusive device 22 within the aneurysm 150 (step 172).

If the energy delivery type is detected as a monopolar type, the electrical energy is not immediately delivered to the implant assembly 14. Instead, another electrical signal is delivered between the proximal end of the pusher member 18 and the ground electrode 36 (step 174), and another electrical parameter is measured in response to the other delivered electrical signal (step 176). If the measured electrical parameter indicates a resistive load between the proximal end of the pusher member 18 and the ground electrode 36 (step 178), electrical energy is delivered to the implant assembly 14 in a monopolar mode to electrolytically detach the vaso-occlusive device 22 within the aneurysm 150 (step 180).

If the electrical parameter measured in response to the electrical signal delivered at step 172 does not indicate either a short circuit or a resistive load; that is, it indicates an open circuit between the two points, or if the other electrical parameter measured in response to the other electrical signal delivered at step 174 does not indicate a resistive load; that is, it indicates an open circuit between the proximal end of the pusher member 18 and the ground electrode 36, a faulty electrical connection is communicated, so that the user can check the electrical connection of the system (step 182), after which the process can be repeated at step 160.

In an alternative method, the energy delivery type is detected by detecting whether the ground electrode 36 is coupled to the power supply 16. In particular, if a coupling between the ground electrode and the power supply is detected, the energy delivery type of the implant assembly 14 will be detected as a monopolar type, in which case, electrical energy will be delivered to the implant assembly 14 in a monopolar mode to electrolytically detach the vaso-occlusive device 22 within the aneurysm 150. If a coupling between the ground electrode and the power supply is not detected, the energy delivery type of the implant assembly 14 will be detected as a bipolar type, in which case, electrical energy will be delivered to the implant assembly 14 in a bipolar mode to electrolytically detach the vaso-occlusive device 22 within the aneurysm 150.

Although particular embodiments of the present invention have been shown and described, it should be understood that the above discussion is not intended to limit the present invention to these embodiments. It will be obvious to those skilled in the art that various changes and modifications may be made without departing from the scope of the present invention. Thus, the present invention is intended to cover alternatives, modifications, and equivalents that may fall within the spirit and scope of the present invention as defined by the claims.

1. A medical system, comprising:
an implant assembly including an elongated pusher member having a proximal end and a distal end, an implantable device mounted to the distal end of the pusher member, and an electrolytically severable joint disposed on the pusher member, wherein the implantable device detaches from the pusher member when the joint is severed; and
a power supply coupled to the implant assembly, the power supply configured for detecting an energy delivery type of the implantable assembly and delivering electrical energy to the implant assembly in a mode corresponding to the detected energy delivery type, thereby electrolytically severing the joint.

2. The medical system of claim 1, wherein the implantable device comprises a vaso-occlusive device.

3. The medical system of claim 1, wherein the detected energy delivery type is one of a monopolar type and a bipolar type.

4. The medical system of claim 1, wherein the power supply is configured for detecting the energy delivery type of the implant assembly by delivering an electrical signal to the implant assembly and measuring an electrical parameter in response to the delivered electrical signal.

5. The medical system of claim 4, wherein the electrical signal is an alternating current signal.

6. The medical system of claim 4, wherein the measured electrical parameter is indicative of an impedance.

7. The medical system of claim 4, wherein the electrical signal is conveyed between two points on the proximal end of the pusher member.

8. The medical system of claim 7, wherein the power supply is configured for detecting that the energy delivery type is a monopolar type if the measured electrical parameter indicates a short circuit between the two points, and for detecting that the energy delivery type is a bipolar type if the measured electrical parameter indicates a finite resistance between the two points.

9. The medical system of claim 8, wherein the power supply is configured for informing a user of a faulty electrical connection if the measured electrical parameter indicates an open circuit between the two points.

10. The medical system of claim 8, wherein, if a monopolar type is detected, the power supply is further configured for delivering another electrical signal between the proximal end of the pusher member and an external ground electrode, measuring another electrical parameter in response to the other delivered electrical signal, and informing a user of a faulty electrical condition if the other measured electrical parameter indicates an open circuit between the proximal end of the pusher member and the external ground electrode.

11. The medical system of claim 1, wherein the power supply is configured for detecting the energy delivery type by detecting whether a ground electrode is mated with the power supply.

12. The medical system of claim 11, wherein the power supply is configured for detecting that the energy delivery type is a monopolar type if a mating of the ground electrode with the power supply is detected, and for detecting that the energy delivery type is a bipolar type if a mating of the ground electrode with the power supply is not detected.

13. A method of performing a medical procedure on a patient, comprising:
delivering an implant assembly within a patient, the implant assembly including an elongated pusher member, an implantable device mounted to a distal end of the pusher member, and an electrolytically severable joint disposed on the pusher member;
coupling the implant assembly to a power supply;
automatically detecting an energy delivery type of the implant assembly; and
delivering electrical energy from the power supply to the implant assembly in a mode corresponding to the detected energy delivery type, thereby electrolytically severing the joint and detaching the implantable device from the pusher member.

14. The method of claim 13, wherein the implantable device is delivered into the patient to occlude a vascular body.

15. The method of claim 13, wherein the detected energy delivery type is one of a monopolar type and a bipolar type.

16. The method of claim 13, wherein the energy delivery type is detected by delivering an electrical signal to the implant assembly and measuring an electrical parameter in response to the delivered electrical signal.

17. The method of claim 16, wherein the electrical signal is an alternating current signal.

18. The method of claim 16, wherein the measured electrical parameter is indicative of an impedance.

19. The method of claim 16, wherein the electrical signal is conveyed between two points on the proximal end of the pusher member.

20. The method of claim 19, wherein the energy delivery type is detected as a monopolar type if the measured electrical parameter indicates a short circuit between the two points, and the energy delivery type is detected as a bipolar type if the measured electrical parameter indicates a resistive load between the two points.

21. The method of claim 20, further comprising informing a user of a faulty electrical connection if the measured electrical parameter indicates an open circuit between the two points.

22. The method of claim 20, further comprising, if a monopolar type is detected, delivering another electrical signal between the proximal end of the pusher member and an external ground electrode, measuring another electrical parameter in response to the other delivered electrical signal, and informing a user of a faulty electrical connection if the other measured electrical parameter indicates an open circuit between the proximal end of the pusher member and the ground electrode.

23. The method of claim 13, wherein the energy delivery type is detected by detecting whether an external ground electrode is coupled to the power supply.

24. The method of claim 23, wherein the energy delivery type is detected as a monopolar type if a coupling between the ground electrode and the power supply is detected, and the energy delivery type is detected as a bipolar delivery mode if a coupling between the ground electrode and the power supply is not detected.

25. A power supply, comprising:
a first negative electrical contact configured for being coupled to an external ground electrode;
a positive electrical contact and a first negative electrical contact configured for being coupled to an implant assembly having a pusher member and an electrolytically detachable implantable device; and
power delivery circuitry configured for being selectively operated in a bipolar delivery mode and a monopolar delivery mode, wherein electrical energy is conveyed between the positive electrical contact and the first negative electrical contact during the monopolar delivery mode, and electrical energy is conveyed between the positive electrical contact and the second negative electrical contact during the bipolar delivery mode.

26. The power supply of claim 25, further comprising a port configured for receiving the proximal end of the pusher member to place the positive electrical contact and the second negative electrical contact into contact with the proximal end of the pusher member.

27. The power supply of claim 25, further comprising:
a switch having an input terminal coupled to a negative terminal of the power delivery circuitry and first and second output terminals respectively coupled to the first and second negative electrical contacts; and
control circuitry configured for selectively operating the switch to couple the input terminal to the first output terminal during the monopolar delivery mode, and to couple the input terminal to the second output terminal during the bipolar delivery mode.

28. The power supply of claim 25, further comprising a power source electrically coupled to the power delivery circuitry.

29. The power supply of claim 25, wherein the power delivery circuitry includes a constant current source configured for conveying the electrical energy.

30. The power supply of claim 25, wherein the electrical energy is conveyed from the power delivery circuitry within the range of 0.1-10 milliampheres.

31. The power supply of claim 25, wherein the electrical energy is conveyed from the power delivery circuitry within the range of 0.1-10 volts.

32. The power supply of claim 25, wherein the electrical energy is direct electrical energy.

33. The power supply of claim 25, further comprising control circuitry configured for determining an energy delivery type of the implant assembly, and for directing the power delivery circuitry to convey the electrical energy between the positive electrical contact and the first negative electrical contact if the determined energy delivery type is a monopolar type, and for directing the power delivery circuitry to convey the electrical energy between the positive electrical contact and the second negative electrical contact if the determined energy delivery type is a bipolar type.

34. The power supply of claim 33, further comprising detection circuitry, wherein the control circuitry is configured for determining the energy delivery type of the implant assembly by directing the detection circuitry to convey an electrical signal between the positive electrical contact and the second negative electrical contact, and measuring an electrical parameter in response to the conveyed electrical signal.

35. The power supply of claim 34, wherein the electrical signal is an alternating current signal.

36. The power supply of claim 34, wherein the measured electrical parameter is indicative of an impedance.

37. The power supply of claim 34, wherein the control circuitry is configured for determining that the energy delivery type is a monopolar type if the measured electrical parameter indicates a short circuit between the positive electrical contact and the second negative electrical contact, and for determining that the energy delivery type is a bipolar type if the measured electrical parameter indicates a resistive load between the positive electrical contact and the second negative electrical contact.

38. The power supply of claim 37, further comprising a status indicator, wherein the control circuitry is configured for directing the status indicator to indicate a faulty electrical connection if the measured electrical parameter indicates an open circuit between the positive electrical contact and the second negative electrical contact.

39. The power supply of claim 37, further comprising a status indicator, wherein, if a monopolar type is determined, the control circuitry is further configured for directing the detection circuitry to convey another electrical signal between the positive electrical contact and the first negative electrical contact, and measuring another electrical parameter in response to the other conveyed electrical signal, and wherein the control circuitry is configured for directing the status indicator to indicate a faulty electrical connection if the other measured electrical parameter indicates an open circuit between the positive electrical contact and the first negative electrical contact.

40. The power supply of claim 39, further comprising a switch having an input terminal coupled to a negative terminal of the detection circuitry, and first and second output terminals respectively coupled to the first and second negative electrical contacts, wherein the control circuitry is configured for selectively operating the switch to couple the input terminal to the first output terminal prior to the conveyance of the other electrical signal, and to couple the input terminal to the second output terminal prior to the conveyance of the electrical signal.

41. The power supply of claim 33, further comprising detection circuitry, wherein the control circuitry is configured for determining the energy delivery type of the implant assembly by directing the detection circuitry to detect a coupling between the ground electrode and the first negative electrical contact, wherein the control circuitry is configured for determining that the energy delivery type is a monopolar type if coupling of the ground electrode to the first negative electrical contact is detected, and for determining that the energy delivery type is a bipolar delivery mode if coupling of the ground electrode to the first negative electrical contact is not detected.

42. A power supply for use with a medical device having an elongated member and a terminal disposed on a proximal end of the elongated member, the power supply comprising:
power delivery circuitry;
an electrical contact electrically coupled to the power delivery circuitry;
a port configured for receiving the proximal end of the elongated member; and
an electrically insulative compliant member configured for urging the electrical terminal into contact with the electrical contact when the proximal end of the elongated member is received into the port.

43. The power supply of claim 42, further comprising another electrical contact electrically coupled to the power delivery circuitry, wherein the compliant member is configured for urging another electrical terminal disposed on the proximal end of the elongated member into contact with the other electrical contact when the proximal end of the elongated member is received into the port.

44. The power supply of claim 42, wherein the power delivery circuitry is configured for delivering electrical energy to the electrical contact within the range of 0.1-10 milliampheres.

45. The power supply of claim 42, wherein the power delivery circuitry is configured for delivering electrical energy to the electrical contact within the range of 0.1-10 volts.

46. The power supply of claim 42, wherein the power delivery circuitry is configured for delivering direct current (DC) electrical energy to the electrical contact.

47. The power supply of claim 42, wherein the port includes a funnel having a large diameter distal portion and a small diameter proximal portion, and the electrical contact is located proximal to the small diameter proximal portion.

48. The power supply of claim 47, wherein the port further includes a cylindrical tube in communication with the small diameter proximal portion of the funnel, and the electrical contact is located proximal to the cylindrical tube.

49. The power supply of claim 42, wherein the compliant member is a compliant pad.

50. The power supply of claim 42, further comprising a gel material disposed within the port that seals the electrical contact from an external environment.

51. The power supply of claim 42, further comprising a power source electrically coupled to the power delivery circuitry.

52. The power supply of claim 42, further comprising an actuator configured for being manipulated by a user to convey electrical energy from the power delivery circuitry to the electrical contact.

53. The power supply of claim 42, further comprising another electrical contact configured for being coupled to a ground electrode.

54. The power supply of claim 42, further comprising a hand-held portable housing in which the port, the power delivery circuitry, the electrical contact, and the compliant member are carried.

55. The power supply of claim 42, further comprising a printed circuit board on which the power delivery circuitry and electrical contact are mounted.

56. A medical system, comprising:
a medical device including an elongated member, an electrical terminal disposed on a proximal end of the elongated member, and at least one operative element disposed on a distal end of the elongated member in electrical communication with the electrical terminal; and
a power supply including power delivery circuitry, an electrical contact, a port in which the proximal end of the elongated member is disposed, and an electrically insulative compliant member that urges the electrical terminal into contact with the electrical contact.

57. The medical system of claim 56, wherein the medical device further includes another electrical terminal disposed on the proximal end of the elongated member, and the at least one operative element is in electrical communication with the other terminal, and wherein the power supply includes another electrical contact electrically coupled to the power delivery circuitry, and the compliant member urges the other electrical terminal into contact with the other electrical contact.

58. The medical system of claim 56, wherein the operative element is an electrolytically severable joint.

59. The medical system of claim 58, wherein the medical device further includes an implantable device configured for detaching from the distal end of the elongated member when the joint is severed.

60. The medical system of claim 59, wherein the implantable device is a vaso-occlusive device.

61. The medical system of claim 56, wherein the port includes a funnel having a large diameter distal portion and a small diameter proximal portion, and the electrical contact is located proximal to the small diameter proximal portion.

62. The medical system of claim 61, wherein the port further includes a cylindrical tube in communication with the small diameter proximal portion of the funnel, and the electrical contact is located proximal to the cylindrical tube.

63. The medical system of claim 56, wherein the compliant member is a compliant pad.

64. The medical system of claim 56, further comprising a gel material disposed within the port that seals the contact from an external environment.

65. The medical system of claim 56, further comprising a power source electrically coupled to the power delivery circuitry.

66. The medical system of claim 56, wherein the power supply further includes an actuator configured for being manipulated by a user to deliver electrical energy from the power delivery circuitry to the electrical contact.

67. The medical system of claim 56, further comprising an external ground electrode, wherein the power supply further includes another electrical contact configured for being coupled to the ground electrode.

68. The medical system of claim 56, wherein the power supply further includes a hand-held portable housing in which the port, the power delivery circuitry, the electrical contact, and the compliant member are carried.

69. The medical system of claim 56, wherein the power supply further includes a printed circuit board on which the power delivery circuitry and electrical contact are mounted.
